SEQUENCE LISTING (15 sequences)

SEQ ID NO: 1 - DNA, 1521 bp, Thermoanaerobacter italicus
gacgaacgctggcggcgtgcctaacacatgcaagtcgagcggtccggcactcaacgtagttgggtggcggatagcggcggacgggtgagtaacgcgtgggcaacctacccttaagaccgggatagcacctcgaaaggggtggtaatactggataagctccttatagggcatcctatggggaggaaagggtagcgcaagctaccgcttaaggatgggcccgcgtcccatcagctagttggtagggtaacggcctaccaaggcgacgacgggtagccggcctgagagggtggtcggccacactgggactgagacacggcccagactcctacgggaggcagcagtggggaatcttgggcaatgggcggaagcctgacccagcgacgccgcgtgggggaggaaggccttcgggttgtaaaccccagtagtgtgggaagaagtggtgacggtaccacacgaaagccccggctaactacgtgccagcagccgcggtaagacgtagggggcgagcgttgtccggaattactgggcgtaaagggcgcgtaggcggtcaatcaagtcaggtgtaaaagacccgggctcaacccgggggtagcacctgaaactggttggctagagggcaggagaggggagtggaattcccggtgtagcggtgaaatgcgtagatatcgggaggaataccagtggcgaaggcgactctctggactggccctgacgctgaggcgcgaaagcgtggggagcgaacaggattagataccctggtagtccacgctgtaaacgatgggtactaggtgtggggcgcggaagcgttccgtgccgtagcgaacgcaataagtaccccgcctggggagtacggccgcaaggttgaaactcaaaggaattgacgggggcccgcacaagcggtggagcatgtggtttaattcgaagcaacgcgaagaaccttaccagggcttgacatgcaggtggtagcgaaccgaaaggtaagcgaccttaccggggggtaaggagcctgcacaggtggtgcatggttgtcgtcagctcgtgtcgtgagatgttgggttaagtcccgcaacgagcgcaacccctgcctctagttgccagcgggtaagccgggcactctagggggactgccgtggaggacacggaggaaggtggggatgacgtcaaatcatcatgccctaaatgccctgggccacacacgtgctacaatggccggtacagagggaagcgaaggagcgatctggagcgaatcccaaaaagccggtccaagttcggattgcaggctgcaactcgcctgcatgaagtcggaatcgctagtaatcgcggatcagcatgccgcggtgaatacgttcccgggccttgtacacaccgcccgtcacaccacgagagcctgcgacacccgaagccggtgacccaacccggcactcaagtggacatgggattatagggaagaaagaagatataatacgtgtccacttgagtgccgggagggagccgtcgaaggtggggcaggtgattggggtg

SEQ ID NO: 2 - DNA, 1521 bp, Thermoanaerobacter italicus
gacgaacgctggcggcgtgcctaacacatgcaagtcgagcggtccggcactcaacgtagttgagtggtggatagcggcggacgggtgagtaacgcgtgggcaacctacccttaagaccgggatagcacctcgaaaggggtggtaatactggataagctccttatagggcatcctatggggaggaaagggtagcgcaagctaccgcttaaggatgggcccgcgtcccatcagctagttggtagggtaacggcctaccaaggcgacgacgggtagccggcctgagagggtggtcggccacactgggactgagacacggcccagactcctacgggaggcagcagtggggaatcttgggcaatgggcggaagcctgacccagcgacgccgcgtgggggaggaaggccttcgggttgtaaaccccagtagtgtgggaagaagtggtgacggtaccacacgaaagccccggctaactacgtgccagcagccgcggtaagacgtagggggcgagcgttgtccggaattactgggcgtaaagggcgcgtaggcggtcaatcaagtcaggtgtaaaagacccgggctcaacccgggggtagcacctgaaactggttggctagagggcaggagaggggagtggaattcccggtgtagcggtgaaatgcgtagatatcgggaggaataccagtggcgaaggcgactctctggactggccctgacgctgaggcgcgaaagcgtggggagcgaacaggattagataccctggtagtccacgctgtaaacgatgggtactaggtgtggggcgcggaagcgttccgtgccgtagcgaacgcaataagtaccccgcctggggagtacggccgcaaggttgaaactcaaaggaattgacgggggcccgcacaagcggtggagcatgtggtttaattcgaagcaacgcgaagaaccttaccagggcttgacatgcaggtggtagcgaaccgaaaggtaagcgaccttaccggggggtaaggagcctgcacaggtggtgcatggttgtcgtcagctcgtgtcgtgagatgttgggttaagtcccgcaacgagcgcaacccctgcctctagttgccagcgggtaagccgggcactctagggggactgccgtggaggacacggaggaaggtggggatgacgtcaaatcatcatgccctaaatgccctgggccacacacgtgctacaatggccggtacagagggaagcgaaggagcgatctggagcgaatcccaaaaagccggtccaagttcggattgcaggctgcaactcgcctgcatgaagtcggaatcgctagtaatcgcggatcagcatgccgcggtgaatacgttcccgggccttgtacacaccgcccgtcacaccacgagagcctgcgacacccgaagccggtgacccaacccggcactcaagtggacatgggattatagggaagaaagaagatataatacgtgtccacttgagtgccgggagggagccgtcgaaggtggggcaggtgattggggtg

SEQ ID NO: 3 - DNA, 200 bp, Thermoanaerobacter italicus
gacgaacgctggcggcgtgcctaacacatgcaagtcgagcggtccggcactcaacgtagttgggtggcggatagcggcggacgggtgagtaacgcgtgggcaacctacccttaagaccgggatagcacctcgaaaggggtggtaatactggataagctccttatagggcatcctatggggaggaaagggtagcgcaagct

SEQ ID NO: 4 - DNA, 400 bp, Thermoanaerobacter italicus
accgcttaaggatgggcccgcgtcccatcagctagttggtagggtaacggcctaccaaggcgacgacgggtagccggcctgagagggtggtcggccacactgggactgagacacggcccagactcctacgggaggcagcagtggggaatcttgggcaatgggcggaagcctgacccagcgacgccgcgtgggggaggaaggccttcgggttgtaaaccccagtagtgtgggaagaagtggtgacggtaccacacgaaagccccggctaactacgtgccagcagccgcggtaagacgtagggggcgagcgttgtccggaattactgggcgtaaagggcgcgtaggcggtcaatcaagtcaggtgtaaaagacccgggctcaacccgggggtagcacctgaa

SEQ ID NO: 5 - DNA, 400 bp, Thermoanaerobacter italicus
actggttggctagagggcaggagaggggagtggaattcccggtgtagcggtgaaatgcgtagatatcgggaggaataccagtggcgaaggcgactctctggactggccctgacgctgaggcgcgaaagcgtggggagcgaacaggattagataccctggtagtccacgctgtaaacgatgggtactaggtgtggggcgcggaagcgttccgtgccgtagcgaacgcaataagtaccccgcctggggagtacggccgcaaggttgaaactcaaaggaattgacgggggcccgcacaagcggtggagcatgtggtttaattcgaagcaacgcgaagaaccttaccagggcttgacatgcaggtggtagcgaaccgaaaggtaagcgaccttaccggggggta

SEQ ID NO: 6 - DNA, 298 bp, Thermoanaerobacter italicus
aggagcctgcacaggtggtgcatggttgtcgtcagctcgtgtcgtgagatgttgggttaagtcccgcaacgagcgcaacccctgcctctagttgccagcgggtaagccgggcactctagggggactgccgtggaggacacggaggaaggtggggatgacgtcaaatcatcatgccctaaatgccctgggccacacacgtgctacaatggccggtacagagggaagcgaaggagcgatctggagcgaatcccaaaaagccggtccaagttcggattgcaggctgcaactcgcctgcatg

SEQ ID NO: 7 - DNA, 223 bp, Thermoanaerobacter italicus
aagtcggaatcgctagtaatcgcggatcagcatgccgcggtgaatacgttcccgggccttgtacacaccgcccgtcacaccacgagagcctgcgacacccgaagccggtgacccaacccggcactcaagtggacatgggattatagggaagaaagaagatataatacgtgtccacttgagtgccgggagggagccgtcgaaggtggggcaggtgattggggtg

SEQ ID NO: 8 - DNA, 200 bp, Thermoanaerobacter italicus
gacgaacgctggcggcgtgcctaacacatgcaagtcgagcggtccggcactcaacgtagttgagtggtggatagcggcggacgggtgagtaacgcgtgggcaacctacccttaagaccgggatagcacctcgaaaggggtggtaatactggataagctccttatagggcatcctatggggaggaaagggtagcgcaagct

SEQ ID NO: 9 - DNA, 1521 bp, Thermoanaerobacter italicus
  variation (63)..(63): n is A, T, C or G, preferably A or G
gacgaacgctggcggcgtgcctaacacatgcaagtcgagcggtccggcactcaacgtagttgngtggnggatagcggcggacgggtgagtaacgcgtgggcaacctacccttaagaccgggatagcacctcgaaaggggtggtaatactggataagctccttatagggcatcctatggggaggaaagggtagcgcaagctaccgcttaaggatgggcccgcgtcccatcagctagttggtagggtaacggcctaccaaggcgacgacgggtagccggcctgagagggtggtcggccacactgggactgagacacggcccagactcctacgggaggcagcagtggggaatcttgggcaatgggcggaagcctgacccagcgacgccgcgtgggggaggaaggccttcgggttgtaaaccccagtagtgtgggaagaagtggtgacggtaccacacgaaagccccggctaactacgtgccagcagccgcggtaagacgtagggggcgagcgttgtccggaattactgggcgtaaagggcgcgtaggcggtcaatcaagtcaggtgtaaaagacccgggctcaacccgggggtagcacctgaaactggttggctagagggcaggagaggggagtggaattcccggtgtagcggtgaaatgcgtagatatcgggaggaataccagtggcgaaggcgactctctggactggccctgacgctgaggcgcgaaagcgtggggagcgaacaggattagataccctggtagtccacgctgtaaacgatgggtactaggtgtggggcgcggaagcgttccgtgccgtagcgaacgcaataagtaccccgcctggggagtacggccgcaaggttgaaactcaaaggaattgacgggggcccgcacaagcggtggagcatgtggtttaattcgaagcaacgcgaagaaccttaccagggcttgacatgcaggtggtagcgaaccgaaaggtaagcgaccttaccggggggtaaggagcctgcacaggtggtgcatggttgtcgtcagctcgtgtcgtgagatgttgggttaagtcccgcaacgagcgcaacccctgcctctagttgccagcgggtaagccgggcactctagggggactgccgtggaggacacggaggaaggtggggatgacgtcaaatcatcatgccctaaatgccctgggccacacacgtgctacaatggccggtacagagggaagcgaaggagcgatctggagcgaatcccaaaaagccggtccaagttcggattgcaggctgcaactcgcctgcatgaagtcggaatcgctagtaatcgcggatcagcatgccgcggtgaatacgttcccgggccttgtacacaccgcccgtcacaccacgagagcctgcgacacccgaagccggtgacccaacccggcactcaagtggacatgggattatagggaagaaagaagatataatacgtgtccacttgagtgccgggagggagccgtcgaaggtggggcaggtgattggggtg

SEQ ID NO: 10 - DNA, 26 bp, Artificial
  Primer sequence
ttccatatctgtaagtcccgctaaag

SEQ ID NO: 11 - DNA, 26 bp, Artificial
  Primer sequence
attaatacaatagttttgacaaatcc

SEQ ID NO: 12 - DNA, 22 bp, Artificial
  Primer sequence
atataaaaagtcacagtgtgaa

SEQ ID NO: 13 - DNA, 22 bp, Artificial
  Primer sequence
cacctattttgcactttttttc

SEQ ID NO: 14 - DNA, 19 bp, Artificial
  Primer sequence
gagtttgatcctggctcag

SEQ ID NO: 15 - DNA, 21 bp, Artificial
  Primer sequence
acggctaccttgttacgactt

(19) United States
(12) Patent Application Publication
(10) Pub. No.: US 2012/0309065 A1
(43) Pub. Date: Dec. 6, 2012

(54) THERMOPHILIC THERMOANAEROBACTER ITALICUS SUBSP. MARATO HAVING HIGH ALCOHOL PRODUCTIVITY

(75) Inventors: Thomas Kvist, Roskilde (DK); Marie Just Mikkelsen, bronshoj (DK); Rasmus Lund Andersen, Kgs. Lyngby (DK)

(73) Assignee: BIOGASOL IPR APS, Ballerup (DK)

(21) Appl. No.: 13/518,379

(22) PCT Filed: Dec. 21, 2010
(86) PCT No.: PCT/EP10/70390

§ 371 Date: Aug. 21, 2012

(30) Foreign Application Priority Data
Dec. 22, 2009 (DK) PA 2009 70297

(51) Int. Cl. C12N 1/20; C12P 7/40; C12P 7/02; C12P 7/26; C12P 3/00; C12N 15/74; C12P 7/06; C12P 7/56; C12P 7/16; C12P 7/18; C12P 7/04; C12P 7/28; C12P 7/52; C12P 7/54; C12P 7/46; C12N 1/21

(52) U.S. Cl. 435/139; 435/252.1; 435/252.3; 435/136; 435/155; 435/148; 435/168; 435/471; 435/161; 435/160; 435/158; 435/157; 435/150; 435/141; 435/140; 435/145

(57) ABSTRACT

Strict anaerobic thermophilic bacterium belonging to the group of Thermoanaerobacter italicus subsp. marato subsp. nov. and mutants and derivatives thereof. The bacterium is particularly suitable for the production of fermentation products such as ethanol, lactic acid, acetic acid and hydrogen from lignocellulosic biomass.

### FIELD OF THE INVENTION

The present invention relates to a novel isolated xylanolytic thermophilic bacterial cell belonging to the group of Thermoanaerobacter italicus, T. italicus subsp. marato, an isolated strain comprising said cell, a method of producing a fermentation product comprising culturing said cell and the use of said cell for the production of a fermentation product.

### BACKGROUND OF THE INVENTION

The industry of producing fermentation products such as ethanol and lactic acid, is facing the challenge of redirecting the production process from fermentation of relatively easily convertible but expensive starchy materials, to the complex but inexpensive lignocellulosic biomass such as wood and residues from agricultural crops, e.g. straw. Unlike starch, which contains homogenous and easily hydrolysed polymers, lignocellulosic biomass contains cellulose (25-53%), hemicellulose (20-35%), polyphenolic lignin (10-25%) and other extractable components. Typically, the first step in utilization of lignocellulosic biomass is a pre-treatment step, in order to fractionate the components of lignocellulosic material and increase their surface area. The pre-treatment method most often used is acid hydrolysis, where the lignocellulosic material is subjected to an acid such as sulphuric acid whereby the sugar polymers cellulose and hemicellulose are partly or completely hydrolysed to their constituent sugar monomers. Another type of lignocellulose hydrolysis is steam explosion, a process comprising heating of the lignocellulosic material by steam injection to a temperature of 190-230° C. A third method is wet oxidation wherein the material is treated with oxygen at 150-185° C. The pre-treatments can be followed by enzymatic hydrolysis to complete the release of sugar monomers. This pre-treatment step results in the hydrolysis of cellulose into glucose while hemicellulose is transformed into the pentoses xylose and arabinose and the hexoses glucose, galactose and mannose. Thus, in contrast to starch, the hydrolysis of lignocellulosic biomass results in the release of pentose sugars in addition to hexose sugars. This implies that useful fermenting organisms need to be able to convert both hexose and pentose sugars to desired fermentation products such as ethanol.

Traditional microorganisms used for e.g. ethanol fermentation, Sacchararomyces cerevisiae and Zymomonas mobilis, do not metabolize pentoses such as xylose and arabinose, and extensive metabolic engineering is thus necessary to improve performance on lignocellulosic substrates. Gram-positive thermophilic bacteria have unique advantages over the conventional ethanol production strains. The primary advantages are their broad substrate specificities and high natural production of ethanol. Moreover, ethanol fermentation at high temperatures (55-70° C.) has many advantages over mesophilic fermentation. One advantage of thermophilic fermentation is the minimisation of the problem of contamination in continuous cultures, since only a few microorganisms are able to grow at such high temperatures in un-detoxified lignocellulose hydrolysate.

Presently, dependent on the pre-treatment method, cellulases and hemicellulases often have to be added to the pre-treated lignocellulosic hydrolysate in order to release sugar-monomers. These enzymes contribute significantly to the production costs of the fermentation products. However, many thermophilic gram-positive strains possess a range of the relevant enzymes and supplementary additions could become less expensive if a thermophilic gram-positive strain is used. Fermentation at high temperature also has the additional advantages of high productivities and substrate conversions and facilitated product recovery.

Lignocellulose hydrolysates contain inhibitors such as furfural, phenols and carboxylic acids, which can potentially inhibit the fermenting organism. Therefore, the organism must also be tolerant to these inhibitors. The inhibitory effect of the hydrolysates can be reduced by applying a detoxification process prior to fermentation. However, the inclusion of this extra process step increases significantly the total cost of the fermentation product and should preferably be avoided. For example, it has been estimated that overliming of willow hydrolysate increase the cost of ethanol production using Escherichia coli by 22% (Von Sivers et al., 1994). It is therefore preferred that the microorganism is capable of producing fermentation products from undetoxified hemicellulose or holocellulose hydrolysates to make it usable in an industrial lignocellulosic-based fermentation process due to the high cost of detoxification process.

It is also particularly advantageous if the potential microorganism is capable of growing on high concentrations of lignocellulosic hydrolysates, i.e. lignocellulosic hydrolysates with high dry-matter content. This is of particular importance when the microorganism is for alcohol production such as ethanol production, since distillation costs increase with decreasing concentrations of alcohol.

U.S. Pat. No. 6,555,350 describes a Thermoanaerobacter strain which is capable of converting pentoses to ethanol. However, this strain has a significant side production of lactic acid and has only been tested in lignocellulosic hydrolysate having a dry-matter concentration of less that 6% wt/wt.

WO2007134607 describes a Thermoanaerobacter strain, BG1, which is capable of converting pentoses to ethanol in high concentrations of un-detoxified hydrolysates.

Kozianowski et al. (1997) discloses a Thermoanaerobacter italicus strain isolated for growth on pectin. There is no evidence presented that the isolated italicus strain can grow well on lignocellulosic hydrolysates or that the genes encoding the acetate kinase and lactate dehydrogenase can be removed by genetic modification.

### OBJECT OF THE INVENTION

It is an object of embodiments of the invention to provide a microorganism which is capable of overcoming the above mentioned obstacles, in particular for the production of ethanol.

### SUMMARY OF THE INVENTION

It has been found by the present inventor(s) that a novel subspecies of Thermoanaerobacter italicus, T. italicus subsp. marato is capable of producing high levels of ethanol and lactic acid while producing a low level of acetic acid.

In one aspect the present invention relates to an isolated Thermoanaerobacter italicus cell comprising a 16S rDNA comprising a sequence selected from the group consisting of: SEQ ID NO 3, SEQ ID NO 4, SEQ ID NO 5, SEQ ID NO 6, SEQ ID NO 7, SEQ ID NO 8, and a combination of any thereof.

In another aspect the present invention relates to an isolated Thermoanaerobacter italicus cell characterized by having a 16S rDNA sequence at least 99.7% identical to SEQ ID NO 9.

In another aspect the present invention relates to an isolated strain comprising a Thermoanaerobacter italicus cell according to any of the preceding aspects.

In another aspect the present invention relates to a method of producing a fermentation product comprising culturing a cell according to the invention or a strain according to the invention under suitable conditions.

In another aspect the present invention relates to a use of an isolated cell according to the invention or a strain according to the invention for the production of a fermentation product selected from the group consisting of an acid, an alcohol, a ketone and hydrogen.

In another aspect the present invention relates to an isolated xylanolytic, thermophilic Thermoanaerobacter italicus cell which produces a fermentation product selected from the group consisting of an acid, an alcohol, a ketone and hydrogen and wherein one or more genes selected from the group consisting of lactate dehydrogenase (EC 1.1.1.27), acetate kinase (EC 2.7.2.1), phosphate acetyltransferase (EC 2.3.1.8),polysaccharase, pyruvate decarboxylase, and alcohol dehydrogenase have been inserted, deleted or substantially inactivated.

### LEGENDS TO THE FIGURES

The invention is disclosed in more detail below with reference to the figures, wherein

FIG. 1 illustrates a phylogenetic tree based on 16S rRNA genes. The bar represents a DNA sequence difference of 0.01 or 1%;

FIG. 2 shows the macroscopic morphology of BG4 and BG10 after 24 hours of growth in minimal medium without agitation;

FIG. 3 shows a microscopic analysis of BG4 and BG10. Sizes of bacteria were measured using Kappa Image Base (Metreo Module). The bar represents a size of 5 μm. The results of size measurement are listed in Table 2;

FIG. 4 shows schematically A: Relative placing of the pta1 and the ak1 genes in the genomes of BG4 and BG10. Primer sites (pta-out-1f and AK-out-1R) resulting in the 2581 bp PCR product are included and the percentage of homology to the genome are shown in parentheses; B: The knock-out construction used to eliminate pta1 and ak1 from the genomes of BG4 and BG10. The arrows (pta1Up) and (ak1down) illustrate the DNA segments constructed to mediate the homologue recombination;

FIG. 5 shows the result of an agarose gel electrophoresis with a 1% agarose gel with PCR products from wt strains and mutants. The arrows points to the mutants, where pta1 and ak1 have been successfully deleted;

FIG. 6 shows the fermentation products from wild type isolate BG4 and the respective mutant BG4pka1. Xylose is shown on the axis to the left. Ethanol, lactic acid (“lactate”) and acetic acid (“acetate”) concentrations are shown on the axis to the right (g/L);

FIG. 7 shows rDNA sequences of various embodiments.

### DETAILED DISCLOSURE OF THE INVENTION

### Definitions

In the present context the term “mutant” is meant to encompass a bacterial cell in which the genome, including one or more chromosomes or potential extra-chromosomal DNA, has been altered at one or more positions, or in which DNA has been added or removed.

In the present context the term “progeny” is meant to encompass the product of bacterial reproduction. A new organism produced by one or more parents.

In the present context the term “propagation product” is meant to encompass the product of bacterial propagation, division or reproduction.

### Specific Embodiments of the Invention

As mentioned above, in one aspect, the present invention relates to an isolated cell comprising a 16S rDNA sequence selected from the group consisting of: SEQ ID NO 3, SEQ ID NO 4, SEQ ID NO 5, SEQ ID NO 6, SEQ ID NO 7, SEQ ID NO 8, and a combination of any thereof. In one embodiment, the 16S rDNA comprises all of SEQ ID NOS 4-7.

In one aspect, the present invention pertains to an isolated Thermoanaerobacter italicus cell having a 16S rDNA sequence at least 99.7% identical to SEQ ID NO 9. Optionally, the 16S rDNA comprises a sequence selected from SEQ ID NO 3, SEQ ID NO 4, SEQ ID NO 5, SEQ ID NO 6, SEQ ID NO 7, SEQ ID NO 8, and any combination thereof.

An embodiment of the invention is an isolated cell comprising a 16S rDNA sequence having the sequence of SEQ ID NO 9. The nucleotides at positions 63 and 68 can be separately selected from of A, T, C and G. In separate embodiments, the nucleotides at positions 63 and 68 are selected from A and G, and from C and T, respectively. Another embodiment of the invention is an isolated cell comprising a 16S rDNA sequence consisting of the sequence of SEQ ID NO 9.

An embodiment of the invention is an isolated cell having a 16S rDNA sequence comprising SEQ ID NO 1.

An embodiment of the invention is an isolated cell having a 16S rDNA sequence comprising SEQ ID NO 2.

An embodiment of the invention is an isolated cell which is BG10 (DSMZ Accession number 23015).

An embodiment of the invention is an isolated cell which is BG4 (DSMZ Accession number 23012).

The invention is based on the isolated bacterial strains BG10 and BG4 that contain 16S rDNA sequences 100% and 99.9% identical to SEQ ID NO 1, respectively (SEQ ID NO 1 and SEQ ID NO 2, respectively). The strains have been deposited in accordance with the terms of the Budapest Treaty on 30 Sep. 2009 with DSMZ—Deutsche Sammlung von Mikroorganismen and Zellkulturen GmbH, Inhoffenstr. 7B, 38124 Braunschweig, Germany under DSMZ accession number DSM 23015 and DSM 23012, respectively.

An embodiment of the invention is an isolated cell comprising a 16S rDNA comprising a sequence selected from the group consisting of: SEQ ID NO 3, SEQ ID NO 4, SEQ ID NO 5, SEQ ID NO 6, and SEQ ID NO 7, wherein SEQ ID NO 3, SEQ ID NO 4, SEQ ID NO 5, SEQ ID NO 6 and SEQ ID NO 7 are consecutive partial sequences of SEQ ID NO 1.

An embodiment of the invention is an isolated cell comprising a 16S rDNA comprising a sequence selected from the group consisting of: SEQ ID NO 4, SEQ ID NO 5, SEQ ID NO 6, SEQ ID NO 7, and SEQ ID NO 8, wherein SEQ ID NO 8, SEQ ID NO 4, SEQ ID NO 5, SEQ ID NO 6, and SEQ ID NO 7 are consecutive partial sequences of SEQ ID NO 2.

T. italicus subsp. marato is capable of growing and producing fermentation products on very high dry-matter concentrations of lignocellulosic hydrolysates. In the present context the term “lignocellulosic hydrolysate” is intended to designate a lignocellulosic biomass which has been subjected to a pre-treatment step whereby lignocellulosic material has been at least partially separated into cellulose, hemicellulose and lignin thereby having increased the surface area of the material. The lignocellulosic material may typically be derived from plant material, such as straw, hay, garden refuse, comminuted wood, fruit hulls and seed hulls.

The pre-treatment method most often used is acid hydrolysis, where the lignocellulosic material is subjected to an acid such as sulphuric acid whereby the sugar polymers cellulose and hemicellulose are partly or completely hydrolysed to their constituent sugar monomers. Another type of lignocellulose hydrolysis is steam explosion, a process comprising heating of the lignocellulosic material by steam injection to a temperature of 190-230° C. A third method is wet oxidation wherein the material is treated with oxygen at 150-185° C. The pre-treatments can be followed by enzymatic hydrolysis to complete the release of sugar monomers. This pre-treatment step results in the hydrolysis of cellulose into glucose while hemicellulose is transformed into the pentoses xylose and arabinose and the hexoses glucose, galactose and mannose. The pre-treatment step may in certain embodiments be supplemented with treatment resulting in further hydrolysis of the cellulose and hemicellulose. The purpose of such an additional hydrolysis treatment is to hydrolyse oligosaccharide and possibly polysaccharide species produced during the acid hydrolysis, wet oxidation, or steam explosion of cellulose and/or hemicellulose origin to form fermentable sugars (e.g. glucose, xylose and possibly other monosaccharides). Such further treatments may be either chemical or enzymatic. Chemical hydrolysis is typically achieved by treatment with an acid, such as treatment with aqueous sulphuric acid, at a temperature in the range of about 100-150° C. Enzymatic hydrolysis is typically performed by treatment with one or more appropriate carbohydrase enzymes such as cellulases, glucosidases and hemicellulases including xylanases.

It was surprisingly found that the bacterial subspecies according to invention is capable of growing in a medium comprising a hydrolysed lignocellulosic biomass material having a dry-matter content of at least 10% wt/wt, such as at least 15% wt/wt, including at least 20% wt/wt, and even as high as at least 25% wt/wt. In such a medium, a bacterial subspecies according to the invention has a volumetric sugar conversion rate of at least 1 g of sugar per liter of fermentation volume per hour (g/l/h), such as at least 2 g/l/h, including at least 5 g/l/h, and even as high as at least 10 g/l/h, under suitable conditions, e.g., those described in Example 6 or 7. As mentioned previously, this has the great advantage that it may not be necessary to dilute the hydrolysate before the fermentation process, and thereby it is possible to obtain higher concentrations of fermentation products such as ethanol, and thereby the costs for subsequently recovering the fermentation products may be decreased. For example the distillation costs for ethanol will increase with decreasing concentrations of alcohol.

The bacterial strain according to the invention is an anaerobic thermophilic bacterium, and it is capable of growing at high temperatures even at or above 70° C. The fact that the strain is capable of operating at this high temperature is of high importance in the conversion of the lignocellulosic material into fermentation products. The conversion rate of carbohydrates into e.g. ethanol is much faster when conducted at high temperatures. For example, the volumetric ethanol productivity of a thermophilic Bacillus is up to ten-fold higher than a conventional yeast fermentation process which operates at 30° C. Consequently, a smaller production plant is required for a given plant capacity, thereby reducing plant construction costs. As also mentioned previously, the high temperature reduces the risk of contamination from other microorganisms, resulting in less downtime, increased plant productivity and a lower energy requirement for feedstock sterilization. The high operation temperature may also facilitate the subsequent recovery of the resulting fermentation products.

Numerous fermentation products are valuable commodities which are utilized in various technological areas, including the food industry and the chemical industry. Presently, the increasing global energy requirements have resulted in increasing focus on alternatives to fossil fuels as energy sources, and ethanol derived from plant materials (bioethanol) has received particular attention as a potential replacement for or supplement to petroleum-derived liquid hydrocarbon products.

The strain according to invention has the potential to be capable of producing a number of different fermentation products, including acids, alcohols, ketones and hydrogen. In one embodiment, the alcohol is selected from ethanol, butanol, propanol, methanol, propanediol and butanediol. In a further embodiment the acid is lactic acid, propionic acid, acetic acid, succinic acid, butyric acid or formic acid and the ketone is acetone.

The T. italicus subsp. marato strains are wild type strains isolated from a reactor containing high amounts of pretreated lignocellulosic biomass, and have several highly advantageous characteristics needed for the conversion of lignocellulosic biomass material. Thus, this base strain possesses all the genetic machinery for the conversion of both pentose and hexose sugars to various fermentation products such as lactic acid and ethanol.

As will be apparent from the below examples, the examination of the complete 16S rDNA sequence showed that the two closely related strains BG4 and BG10 are both related to Thermoanaerobacter italicus although the 16rDNA sequences clearly places them in a separate subspecies.

The following examples will show that members of subspecies marato have very different morphology. It is contemplated that very elongated members of the subfamily, such as BG10, may advantageously be used in fermentation concepts where a fast sedimentation is required. Such concepts would include concepts where a concentration of the cells is provided without the use, or limiting the use, of filtration or centrifugation equipment.

It is demonstrated in the following examples, that members of T. italicus subsp. marato such as BG4 and BG10 in advantageous embodiments may be modified in order to obtain mutants or derivatives with improved characteristics. Thus, in one embodiment there is provided a bacterial strain according to the invention which is a variant or mutant of T. italicus subsp. marato wherein one or more genes have been inserted, deleted or substantially inactivated.

The variant or mutant is typically capable of growing in a medium comprising a hydrolysed lignocellulosic biomass material having a dry-matter content of at least 10%, at least 15, or at least 25% wt/wt at temperatures at or above 40° C. In one embodiment, the mutant is capable of very high productivity of ethanol, e.g., at least 1.5 g/L/h, at least 1.9 g/L/h, or at least about 2 g/L/h, under suitable conditions, such as those described in Examples 6 or 7.

In another embodiment, there is provided a process for preparing such a variant or mutant of T. italicus subsp. marato, wherein one or more genes are inserted, deleted or substantially inactivated as described herein.

As shown in the following examples, deletion of the genes encoding phosphotransacetylase (EC 2.3.1.8) and acetate kinase (EC 2.7.2.1) was found not to be possible in the related species Thermoanaerobacter mathranii BG1 as disclosed in WO2007/134607. The antibiotic resistance marker used was found to integrate into a different position in the chromosome, leaving acetic acid production unaffected. Without being bound to theory, the genes seem to be essential in BG1 or, the deletion is too inhibitory to allow significant growth. Surprisingly, when the same deletion was attempted in the T. italicus subsp. marato, represented by strains BG4 and BG10, the antibiotic resistance marker was successfully integrated and replaced the pta1 and ak1 genes. Accordingly, the resulting mutant strains showed almost no acetic acid production.

An embodiment of the invention is an isolated cell, which is BG10pka (DSMZ Accession number 23216) or BG4pka (DSMZ Accession number 23013).

Therefore, it is contemplated that the subspecies in accordance with the invention may be a modified version of a member of the subspecies, wherein the gene encoding phosphotransacetylase (EC 2.3.1.8) and/or acetate kinase (EC 2.7.2.1) has been inactivated by the deletion of said gene, or wherein the gene has been substantially inactivated by the mutation, deletion or insertion of one or more amino acids in the gene.

It has been found that the ethanol producing capability of the related species T. mathranii BG1 may be significantly increased by inactivating the gene encoding lactate dehydrogenase (LDH) (EC 1.1.1.27).

Therefore, it is contemplated that the subspecies in accordance with the invention may be a modified version of a member of the subspecies, wherein the gene encoding lactate dehydrogenase (LDH) (EC 1.1.1.27) has been inactivated by the deletion of said gene, or wherein the gene has been substantially inactivated by the mutation, deletion or insertion of one or more amino acids in the gene.

An embodiment of the invention is an isolated cell, which is BG10XL (DSMZ Accession number 23017) or BG4XL (DSMZ Accession number 23014).

It is also contemplated that the subspecies according to the invention may be a modified member of the subspecies, wherein both the genes encoding lactate dehydrogenase (LDH) (EC 1.1.1.27) and the genes encoding phosphotransacetylase (EC 2.3.1.8) and/or acetate kinase (Ec 2.7.2.1) have been inactivated by the deletion of said gene, or wherein the gene has been substantially inactivated by the mutation, deletion or insertion of one or more amino acids in the gene.

Further provided by the invention is a process for preparing such a modified member of the subspecies comprising inactivating the gene encoding phosphotransacetylase (EC 2.3.1.8) acetate kinase (EC 2.7.2.1), and/or lactate dehydrogenase (LDH) (EC 1.1.1.27) by the deletion of said gene, or by substantially inactivating the gene by the mutation, deletion or insertion of one or more amino acids in the gene. In one embodiment, the process comprises inactivating or substantially inactivating both a gene encoding lactate dehydrogenase (LDH) (EC 1.1.1.27) and a gene encoding phosphotransacetylase (EC 2.3.1.8) and/or acetate kinase (EC 2.7.2.1).

As mentioned above, T. italicus subsp. marato possesses the genetic machinery to enable it to convert both hexose sugars and pentose sugars to a range of desired fermentation products, including ethanol.

However, it may for certain embodiments be desired to insert one or more additional genes into the strain according to the invention. Thus, in order to improve the yield of the specific fermentation product, it may be beneficial to insert one or more genes encoding a polysaccharase into the strain according to the invention. Hence, in specific embodiments there is provided a strain and a process according to the invention wherein one or more genes encoding a polysaccharase which is selected from cellulases (such as EC 3.2.1.4); beta-glucanases, including glucan-1,3 beta-glucosidases (exo-1,3 beta-glucanases, such as EC 3.2.1.58), 1,4-beta-cellobiohydrolases (such as EC 3.2.1.91) and endo-1,3(4)-beta-glucanases (such as EC 3.2.1.6); xylanases, including endo-1,4-beta-xylanases (such as EC 3.2.1.8) and xylan 1,4-beta-xylosidases (such as EC 3.2.1.37); pectinases (such as EC 3.2.1.15); alpha-glucuronidases, alpha-L-arabinofuranosidases (such as EC 3.2.1.55), acetylesterases (such as EC 3.1.1.-), acetylxylanesterases (such as EC 3.1.1.72), alpha amylases (such as EC 3.2.1.1), beta-amylases (such as EC 3.2.1.2), glucoamylases (such as EC 3.2.1.3), pullulanases (such as EC 3.2.1.41), beta-glucanases (such as EC 3.2.1.73), hemicellulases , arabinosidases, mannanases including mannan endo-1,4-beta-mannosidases (such as EC 3.2.1.78) and mannan endo-1,6-alpha-mannosidases (such as EC 3.2.1.101), pectin hydrolases, polygalacturonases (such as EC 3.2.1.15), exopolygalacturonases (such as EC 3.2.1.67) and pectate lyases (such as EC 4.2.2.10), are inserted.

Depending on the desired fermentation product, it is contemplated that in certain embodiments it is useful to insert a gene encoding a pyruvate decarboxylase (such as EC 4.1.1.1) or to insert a heterologous alcohol dehydrogenase (such as EC 1.1.1.1, EC 1.1.1.2, EC 1.1.1.71, or EC 1.1.99.8) or to up-regulate an already existing alcohol dehydrogenase.

In accordance with the invention, a method of producing a fermentation product comprising culturing a strain according to the invention under suitable conditions is also provided.

The strain according to the invention is a strict anaerobic microorganism, and hence it is preferred that the fermentation product is produced by a fermentation process performed under strict anaerobic conditions. Additionally, the strain according to invention is a thermophillic microorganism, and therefore the process may perform optimally, when it is operated at temperature in the range of about 40-95° C., such as the range of about 50-90° C., including the range of about 60-85° C., such as the range of about 65-75° C.

For the production of certain fermentation products, it may be useful to select a specific fermentation process, such as batch fermentation process, including a fed batch process or a continuous fermentation process. Also, it may be useful to select a fermentation reactor such as an immobilized cell reactor, a fluidized bed reactor or a membrane bioreactor.

In accordance with the invention, the method is useful for the production of a wide range of fermentation products including acids, alcohols, ketones and hydrogen. Thus fermentation products such as ethanol, butanol, propanol, methanol, propanediol, butanediol, lactic acid, propionic acid, acetic acid, succinic acid, butyric acid, formic acid and acetone may be produced in accordance with the invention.

The invention will now be further described in the following non-limiting examples and figures.

### Example 1

### Materials and Methods

The following materials and methods were applied in the below Examples:

### Cultivation and Isolation

Isolations and subsequent cultivation in axenic cultures were conducted from continuous enrichment fermentations designed to have an increasing elevated inhibitor concentrations (ref reactor design). Enrichment reactors were inoculated with a complex mix of environmental samples and were run with pretreated wheat straw (ref pretreatment). All strains were isolated at 70° C. anaerobically and were finally cultivated in basal anaerobic minimal medium (BA) (Larsen et al. 1997) supplemented with 2 g/L yeast extract. Final isolations were carried out on solid surface cultivation using Hungate Roll Tubes (Hungate 1969) on the same medium with the addition of phytagel. Solid surface isolations were repeated twice following the first isolation in order to ensure that isolation cultures were axenic.

### Enzymes and Reagents

If not stated otherwise enzymes were supplied by MBI Fermentas (Germany) and used according to the suppliers recommendations. Chemicals were of molecular grade and were purchased from Sigma-Aldrich Sweden AB.

### Phenotype

The phenotypes of the isolated cultures were analyzed at the macroscopic and microscopic level. Images of batch cultures were taken after 24 hrs growth at 70 ° C. using a Canon digital camera (specs). Microscopic images are taken using a Leica DMIRBE inverted microscope with a Kappa DX20H camera for image documentation. Measurements of the individual bacterial cell sizes were conducted using Kappa Metreo module.

### HPLC

Sugars and fermentation products were quantified by HPLC-RI using a Dionex Ulitimate 3000 (Dionex corp.) fitted with an Aminex HPX-87H column (300×7.8 mm) (Bio-Rad Laboratories, CA, USA) combined with a Cation H refill cartridge (30×4.6 mm) (Bio-Rad Laboratories, CA, USA). The analysts were separated isocratically with 4 mM H₂SO₄ and at 60° C.

1 ml sample was used for HPLC analysis. 20 μl H₂SO₄ were added to the sample followed by 30 sec vortex. Sample was centrifuged at 14.000 G for 10 minutes, and 500 μl of the supernatant was used for HPLC analysis.

### PTA and AK Removal

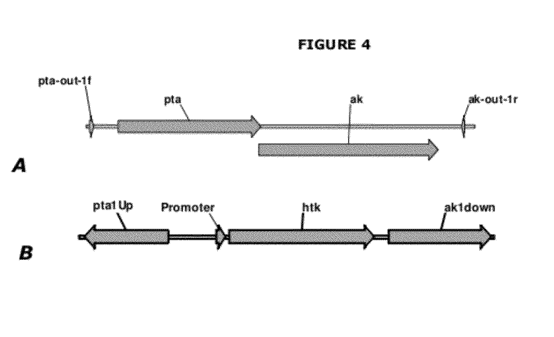

A dual elimination of both phosphotransacetylase (PTA) and acetate kinase (AK) was performed on isolates BG4 and BG10. The genetic knock-out cassette described is illustrated in FIG. 4. The integration was designed as a double cross-over using homologous recombination of transformed DNA. Successful integration of the knock-out cassette was selected for by subsequent cultivation in 100 μg/ml kanamycin in the above mentioned growth medium. Integration was verified by PCR using primers targeting DNA sequence flanking the inserted DNA. A successful integration was evaluated by PCR using PTA-out-1f (5′-ggt aaa ggt gtc cgt agt gaa aag g-3′) and AK-out-1r primers (5′-cca ata ctc tca acg tct tcc ac-3′) resulting in PCR products of 1928 by for the successful integration. The PCR products of the corresponding wild type are of 2581 bp.

### Ldh Removal

A single gene knock out construct, p3TPKc2 contains 1) a DNA fragment upstream of the l-ldh gene of BG4 and BG10, amplified using primers ldhup1f (5′-TTCCATATCTGTAAGTCCCGCTAAAG-3′; SEQ ID NO:10) and ldhup2r (5′-ATTAATACAATAGTTTTGACAAATCC-3′; SEQ ID NO:11), 2) a gene encoding a highly thermostable kanamycin resistance amplified from pUC18HTK (Hoseki et al. 1999), and 3) a DNA fragment downstream of the l-ldh gene of BG4 and BG10 amplified using primers ldhdown3f (5′-ATATAAAAAGTCACAGTGTGAA-3′; SEQ ID NO:12) and ldhdown4r (5′-CACCTATTTTGCACTTTTTTTC-3′; SEQ ID NO:13). The p3TPKc2 was linearised and chemically transformed into BG4 and BG10.

Phylogenetic Analysis of 16S rRNA Genes

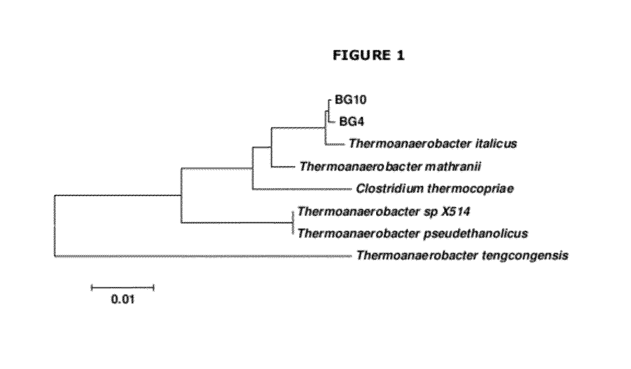

1,5 ml culture was harvested and used as template for DNA extraction. DNA was extracted using Genomic Mini (Aabiot, Poland) as described by the manufacturer. Approximately 1500 by rRNA gene was amplified by PCR using proofreading Pwo-polymerase (A&A biotech, Poland) and pre-phosphorylated primers B1 (5′-PHO-GAG TTT GAT CCT GGC TCA G-3′; SEQ ID NO:14) and B2 (5′-PHO-ACG GCT ACC TTG TTA CGA CTT-3′; SEQ ID NO:15). The generated PCR-products were excised from a 1% agarose gel and extracted using QiaExII gel extraction kit. The extracted products were subsequently cloned into Ecl136II digested pUC19 vector treated with SAP (Shrimp Alkaline Phosphatase). 30 clones from each isolate were analyzed by restriction fragment length polymorphism (RFLP) using restriction enzyme MboI (′GATC) and BsuRI (GG′CC). Representatives from each unique restriction band pattern were sequenced forward and reverse by Euofins-MWG (Germany). Sequences were trimmed in order to eliminate vector and primary PCR primer sequences. Forward and reverse reads were subsequently assembled using assembly in VectorNTI (Invitrogen). The closest relatives to each of the clones were detected using the “Sequence Match” function (Cole et al. 2003) and “Blastn” from NCBI (Altschul et al. 1990). Alignment was carried out using ClustalW (Chenna et al. 2003) and the phylogenetic tree was constructed using Software MEGA4 (Kumar et al. 2001) based on neighbor-joining (Jukes-Cantor, Gaps: Complete deletion) with Thermoanaerobacter tencongensis as outgroup. The distance matrix based on nucleotide comparison is shown below in Table 1. The associated phylogeny is illustrated in FIG. 1.

TABLE 1

| | | | | | Thermo- | | Thermo- |
|---|---|---|---|---|---|---|---|
| | | | Clostridium | Thermoanaerobacter | anaerobacter | Thermoanaerobacter | anaerobacter |
| | BG10 | BG4 | thermocopriae | italicus | mathranii | sp X514 | pseudethanolicus |
| BG10 | | | | | | | |
| BG4 | 0.001 | | | | | | |
| Clostridium | 0.030 | 0.031 | | | | | |
| thermocopriae | | | | | | | |
| Thermoanaerobacter | 0.004 | 0.004 | 0.031 | | | | |
| italicus | | | | | | | |
| Thermoanaerobacter | 0.013 | 0.014 | 0.019 | 0.016 | | | |
| mathranii | | | | | | | |
| Thermoanaerobacter | 0.041 | 0.042 | 0.046 | 0.043 | 0.039 | | |
| sp X514 | | | | | | | |
| Thermoanaerobacter | 0.041 | 0.042 | 0.046 | 0.043 | 0.039 | 0.000 | |
| pseudethanolicus | | | | | | | |
| Thermoanaerobacter | 0.091 | 0.091 | 0.095 | 0.095 | 0.091 | 0.087 | 0.087 |
| tengcongensis | | | | | | | |

16S rDNA distance matrix showing fraction of different base pairs to total base pairs.

### Batch Experiments.

Wild type isolates and genetically modified strains (pka mutations) were analyzed in triplicate, and the performance of the mutants was compared to the corresponding wild types. The growth was monitored in 10 ml batch cultures with 5 g/L Xylose without antibiotic selection. Samples were monitored for a 72 hrs period.

### Continuous Fermentation Using Thermoanaerobacter BG10XL

The reactor was a water-jacketed glass column with a working volume of 200 mL. All pumps and pH-measuring were controlled by an Applikon Bio Console ADI 1025 system. The influent entered via a hose pump from the bottom of the reactor. A recirculation flow of 1.6 m/h was achieved using two identical pump heads running with staggered rollers providing low pulsation. The pH was maintained at 7.3 by addition of NaOH (1 or 2 M). The continuous fermentation was performed at 70° C. by external heating and recirculation of hot water in the glass jacket. The entire reactor system, including tubing and recirculation reservoir was autoclaved at 121° C. for 30 minutes. Liquid samples for HPLC were taken from a sampling port located in the recirculation stream almost daily.

The sterilized reactor was inoculated with Thermoanaerobacter BG10XL (DSM 23017) and was operated in batch for 24 hours before the influent was turned on and continuous operation initiated. The reactor was operated as a continuous fluidized bed reactor with decreasing hydraulic retention time from 25 to 6 hours.

### Example 2

### Isolation of BG4 and BG10

The two new strains were isolated from continuous reactor containing pretreated and enzymatically hydrolyzed wheat straw. The resulting medium contain high amounts of fermentation inhibitors and the organism which has the higher tolerance to these inhibitors will therefore grow faster and will eventually take over the population. The reactor was initially inoculated with Thermoanaerobacter mathranii BG1. After initial adaptation the reactor was repeatedly inoculated with environmental samples from hot environments as well as with soil and compost samples. After four months of continuous operation, single strains were isolated from the reactor. Surprisingly, a large proportion of the isolates belonged to a new subspecies related to Thermoanaerobacter italicus. This indicates that the new isolates are even more resistant to the fermentation inhibitors than T. mathranii and will therefore be able to grow faster in high concentrations of lignocellulosic hydrolysate. Two of the isolates, BG4 and BG10, produced high amounts of ethanol (FIG. 6).

### Example 3

### Phenotype of BG4 and BG10

The phenotype of T. italicus subsp. marato BG4 and BG10 prove to be different at both the macroscopic and microscopic level. Cultures of BG4 resemble those seen in other well characterized Thermoanaerobacter species (Larsen et al., 1997) where bacterial cells remain in suspension during fermentation. This observation is backed up by the microscope images where BG4-cells are recognized as straight rods ranging in size from 2μm to 4 μm. Measurement data of the microscopic measurements are listed in Table 2 below.

TABLE 2

| | Number | | |
|---|---|---|---|
| Strain | measured | Average | Stdev |
| BG10 | 8 | 189.7 μm | 94.4 μm |
| BG4 | 57 | 3.3 μm | 0.8 μm |

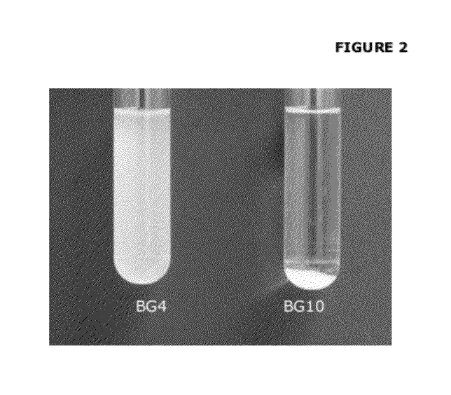

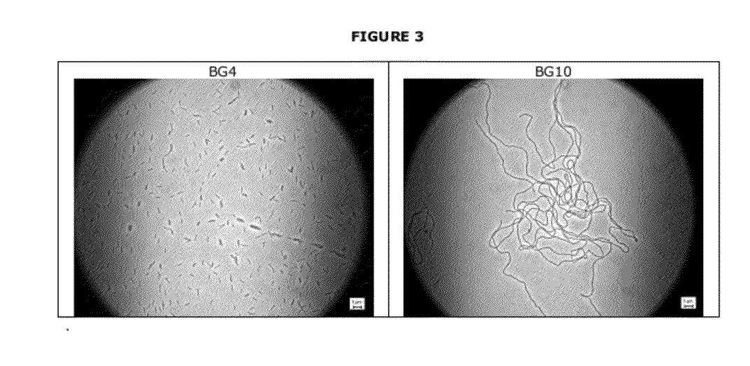

Surprisingly however, both macroscopic and microscopic phenotype differs fundamentally in the isolate BG10 as compared to that of BG4. Batch cultures of BG10 form a “pellet-like” substance at the bottom of the culture during fermentation. Although the optical density of BG4 and BG10 are in the same range when suspended in culture medium, the images illustrated in FIG. 2 clearly show a phenotypic difference. BG4 cells remain in suspension during cultivation, whereas the cells in BG10 are forming a “pellet-like” substance at the bottom of the incubation tube. In addition, different size measurements result from the measurements of BG10 as compared to those of BG1 emphasizes the phenotypic difference. The size of BG10 cells are on average 189.7 μm or 57.5 times longer than those of BG4, probably causing the cells to sediment into the “pellet like structure”.

### Example 4

### Phylogeny

Sequencing of 16S rDNA from BG4 and BG10 revealed that Both BG4 and BG10 had (at least) one copy of a 16s rRNA operon which was most closely related to Thermoanaerobacter italicus in the available public databases. However, the 16S rDNA genes from both isolated strains (BG10) and (BG4) have multiple sequence differences as compared to the sequence from Thermoanaerobacter italicus (submitted 15-NOV-1999 by Sproeer C., Molecular Systematics, DSMZ, Mascheroder Weg 1B, D-38124 Braunschweig, GERMANY, by direct submission to Genbank under accession number AJ250846.1), corresponding to 0.4% of the sequence.

The 16S rDNA sequences of the BG4 and BG10 strains have only 2 by difference in 1524 by (0.1%) and the two strains are therefore proposed to belong to the same subspecies, Thermoanaerobacter italicus subsp. marato.

As a distance matrix does not take into account differences which occur in areas with gaps, a true difference between two different isolates is equal to or above the value of the distance matrix.

### Example 5

### Pta1 and Ak1 Gene Deletion.

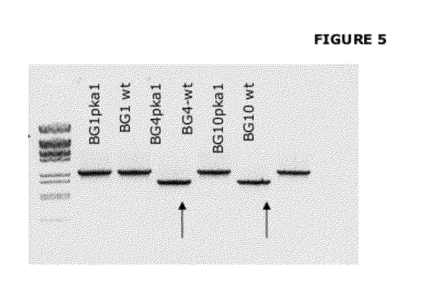

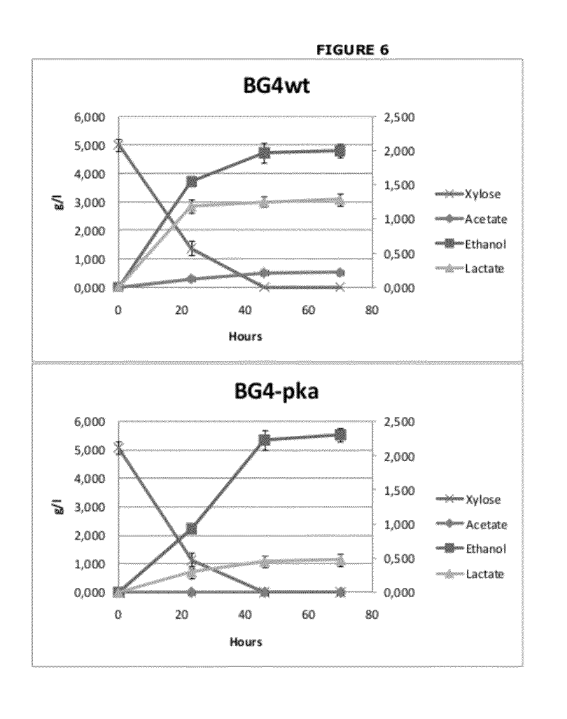

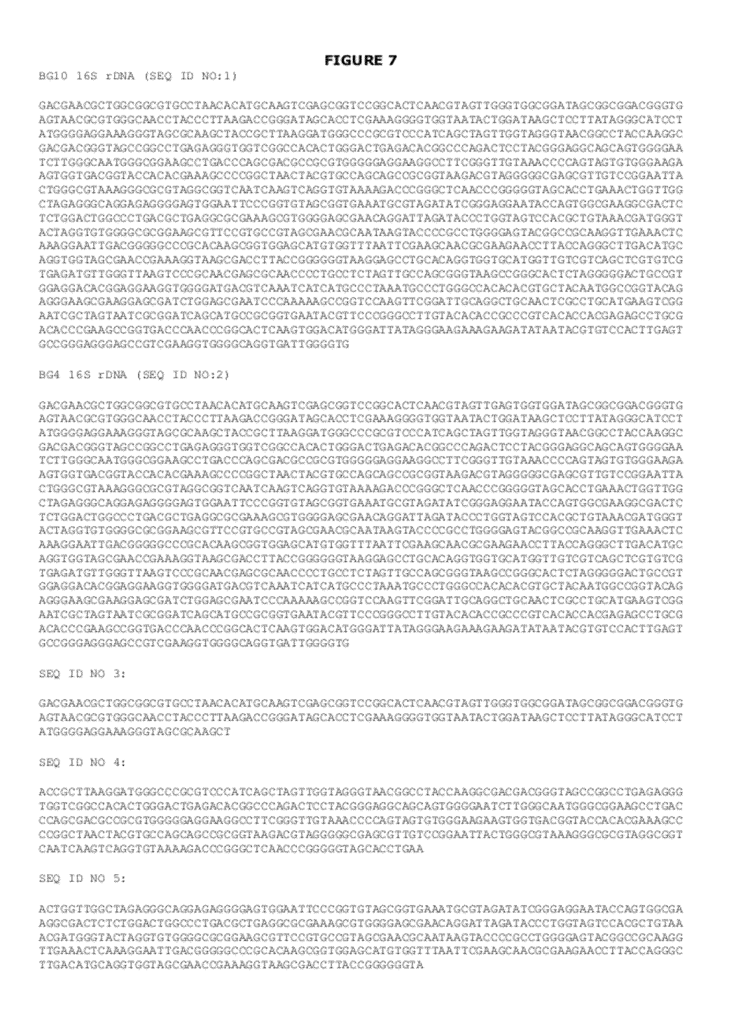

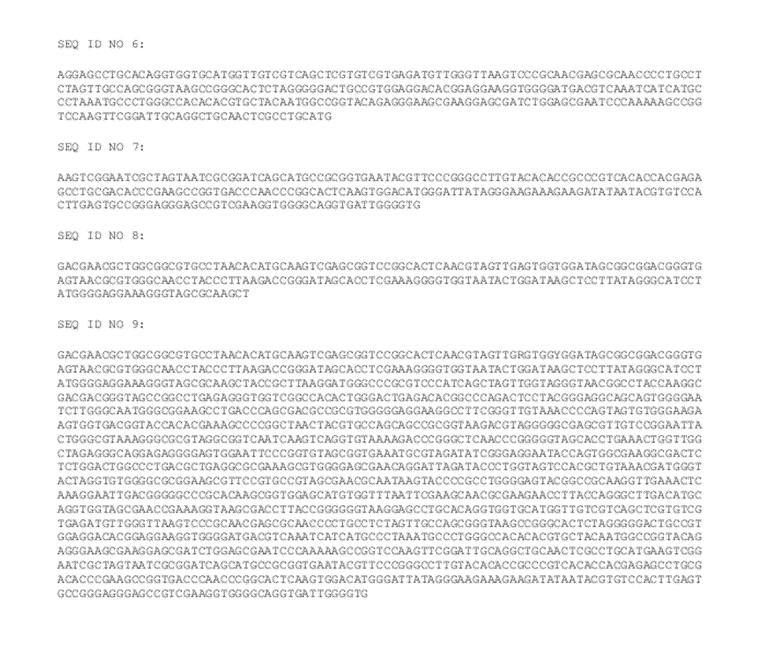

The phophotransacetylase (PTA) and acetate kinase (AK) enzymes are necessary for production of acetic acid. Acetic acid is an unwanted byproduct in ethanol production and the production can therefore favorably be removed. The pta1 and ak1 genes encoding PTA and AK were successfully removed from both BG4 and BG10. BG4 and BG10 strains with pta1 and ak1 gene deletion, i.e. BG4pka and BG10pka, have been deposited in accordance with the terms of the Budapest Treaty on 30 Sep. 2009 and 18 Dec. 2009, respectively, with DSMZ—Deutsche Sammlung von Mikroorganismen and Zellkulturen GmbH, Inhoffenstr. 7B, 38124 Braunschweig, Germany under DSMZ accession number DSM 23013 and DSM 23216, respectively. The resulting PCR product from a successful insertion of the knock-out cassette is 1928 bp. The resulting PCR from the wild type is 2581 bp. Both PCRs, the successfully inserted knock-out and the corresponding wild type product are illustrated in FIG. 5.

Surprisingly, the same figure also clearly illustrates, that the introduced genetic knock-out is not implementable in the related Thermoanaerobacter mathranii strain BG1. However, the transformed cells do express a genetic resistance towards kanamycin, and have therefore incorporated the HTK gene.

These data are supported by the HPLC analysis of each of the isolates and the associated generated mutants (FIG. 6, Table 3 below). Neither BG4 nor BG10 produce acetic acid in the absence of the pta1 and ak1 genes. Thermoanaerobacter mathranii BG1 does not prove to have any detectable reduction in acetic acid production as a result of replacement of the pta1 and ak1 genes with the kanamycin resistance gene. All wildtype (wt) isolates, including BG1, produce acetic acid if the pta1 and ak1 genes are not removed.

The lactic acid production is decreased in both BG4 and BG10 when the pta1 and ak1 genes are removed. BG4pka has an increased ethanol yield as compared to BG4, whereas the ethanol production is unchanged when pta1 and ak1 genes are removed from BG10.

TABLE 3

| | Acetic | | Lactic |
|---|---|---|---|
| | acid | Ethanol | acid |
| BG4 | 0.22 | 2.00 | 1.29 |
| BG4XL | 0.24 | 2.00 | 0.00 |
| BG4pka | 0.00 | 2.31 | 0.48 |
| BG10 | 0.20 | 1.23 | 3.31 |
| BG10XL | 0.33 | 1.99 | 0.00 |
| BG10pka | 0.00 | 1.20 | 1.14 |
| BG1 | 0.40 | 1.05 | 0.10 |
| BG1pka | 0.41 | 1.22 | 0.04 |

The table shows the fermentation products (in g/L) from the two isolates BG4 and BG10 and the respective mutants after 70 hours of growth. For comparison the same values are shown for Thermoanaerobacter mathranii BG1 (WO2007134607).

### Example 6

The lactate dehydrogenase is the major enzyme in production of lactic acid in species such as Thermoanaerobacter. The lactate dehydrogease was deleted from both BG4 and BG10 As shown in table 3, deletion of the lactate dehydrogenase from T. italicus subsp. marato strains BG4 and BG10 resulted in strains that do not produce lactic acid (BG4XL and BG10XL). For both strains acetic acid production was only increased insignificantly. BG4XL and BG10XL strains have been deposited in accordance with the terms of the Budapest Treaty on 30 Sep. 2009 with DSMZ—Deutsche Sammlung von Mikroorganismen and Zellkulturen GmbH, Inhoffenstr. 7B, 38124 Braunschweig, Germany under DSMZ accession number DSM 23014 and DSM 23017, respectively.

It is therefore likely that deletion of pta1 and ak1 genes in combination with deletion of the lactate dehydrogenase gene will result in strains that produce only or almost only ethanol.

### Continuous Fermentation of Corn Cob Sugars Using BG10XL

Influent medium: corn cob was pretreated using catalyzed steamexplosion and centrifuged. The liquid supernatants containing the C5 sugars was added nutrients and the liquor was sterile filtrated. The final content of sugars in the influent medium was: cellobiose, 1.24 g/L; glucose, 4.51 g/L; xylose, 44.20 g/L; arabinose, 4.55 g/L.

A continuous reactor was set up as described in materials and methods. The cells were allowed to immobilize on the reactor carrier material and the reactor was then set to continuous operation. The hydraulic retention time was gradually decreased until a sugar conversion below 80% was found. At the lowest retention time a very high productivity of more than 2 g of ethanol per liter of reactor volume per hour, was recorded (FIG. 6, Table 4 below). This surprisingly high productivity has not previously been recorded for any thermophilic bacterial strain in concentrated lignocellulosic material. Two reports on fermentation of undetoxified lignocellulosic hydrolysates with T. mathranii BG1L1 have shown maximal ethanol productivities of below 0.2 g/L/h, which is less than 10% of the ethanol productivity reported here (Georgieva and Ahring 2007; Georgieva et al. 2008). An ethanol productivity of 2.1 g/l/h corresponds to a sugar conversion rate of 5.0 at an ethanol yield of 0.42 g/g.

TABLE 4
Continuous ethanol fermentation using BG10XL.
Fermentation summary.

| Ethanol | Ethanol | Max | Total | Operation |
|---|---|---|---|---|
| productivity¹ | yield² | DM³ | sugar | time |
| g/L/h | g/g | % | % | days |
| 2.1 | 0.41-0.43 | 12 | 80-90 | 44 |

¹Volumetric productivity expressed in g of ethanol per l of total reactor volume per hour.
²Ethanol yield expressed in g per g consumed sugar.
³Maximal drymatter of added biomass just after pretreatment.

The ethanol productivity relates directly to the size of the reactor: A tenfold higher productivity results in a tenfold reduction in necessary reactor size, meaning a significant reduction in capital investment.

### Example 7

### Continuous Fermentation of Wheat Straw Sugars Using BG10XL

Influent medium: wheat straw was pretreated using catalyzed steam-explosion and centrifuged. The liquid supernatants containing the C5 sugars were added nutrients, pH was adjusted to 3 and the liquor was sterile filtrated. The content of sugars in the influent medium was: glucose, 2.5 g/L; xylose, 28 g/L; arabinose, 3.9 g/L, corresponding to 13.8% DM before centrifugation.

The continuous reactor system was used to test ethanol production of Thermoanaerobacter BG10XL (DSM 23017) on a high dry matter material is identical to the system described in example 1. During the fermentation the dry matter content was increased from 0% to 13.8%. After reaching 13.8% dry matter (highest possible in the pretreatment reactor) additional xylose was added, finally reaching a xylose concentration equivalent to around 28% DM (69 g/l xylose). Results obtained during fermentation are listed in table 5.

TABLE 5
Table 5. Fermentation summary for continuous reactor.

| | | | | Total | |
|---|---|---|---|---|---|
| Ethanol | Ethanol | Ethanol | Max | sugar | Operation |
| Conc¹ | prod.² | yield³ | DM⁴ | conversion | time |
| g/l | g/l/h | g/g | % | % | Days |
| 28.7 | 1.1 | 0.42 | 13.8-28 | 88-95 | 61 |

¹Corrected for addition of NaOH and for evaporation.
²Volumetric productivity expressed in g of ethanol per l of total reactor volume per hour.
³Ethanol yield expressed in g per g consumed sugar.
⁴Maximal dry-matter of added biomass just after pretreatment.

LIST OF REFERENCES

ALTSCHUL, S. F., GISH, W., MILLER, W., MYERS, E. W. AND LIPMAN, D. J. (1990) BASIC LOCAL ALIGNMENT SEARCH TOOL. JMOLBIOL 215, 403-410.

CHENNA, R., SUGAWARA, H., KOIKE, T., LOPEZ, R., GIBSON, T. J., HIGGINS, D. G. AND THOMPSON, J. D. (2003) MULTIPLE SEQUENCE ALIGNMENT WITH THE CLUSTAL SERIES OF PROGRAMS. NUCLEIC ACIDS RES 31, 3497-3500.

COLE, J. R., CHAI, B., MARSH, T. L., FARRIS, R. J., WANG, Q., KULAM, S. A., CHANDRA, S., MCGARRELL, D. M., SCHMIDT, T. M., GARRITY, G. M. AND TIEDJE, J. M. (2003) THE RIBOSOMAL DATABASE PROJECT (RDP-II): PREVIEWING A NEW AUTOALIGNER THAT ALLOWS REGULAR UPDATES AND THE NEW PROKARYOTIC TAXONOMY. NUCLEIC ACIDS RES 31, 442-443.

GEORGIEVA, T. I. AND AHRING, B. K. (2007) EVALUATION OF CONTINUOUS ETHANOL FERMENTATION OF DILUTE-ACID CORN STOVER HYDROLYSATE USING THERMOPHILIC ANAEROBIC BACTERIUM THERMOANAEROBACTER BG1L1. APPL MICROBIOL BIOTECHNOL 77, 61-68.

GEORGIEVA, T. I., MIKKELSEN, M. J. AND AHRING, B. K. (2008) ETHANOL PRODUCTION FROM WET-EXPLODED WHEAT STRAW HYDROLYSATE BY THERMOPHILIC ANAEROBIC BACTERIUM THERMOANAEROBACTER BG1L1 IN A CONTINUOUS IMMOBILIZED REACTOR. APPL BIOCHEM BIOTECHNOL 145, 99-110.

HOSEKI, J., YANO, T., KOYAMA, Y., KURAMITSU, S. AND KAGAMIYAMA, H. (1999) DIRECTED EVOLUTION OF THERMOSTABLE KANAMYCIN-RESISTANCE GENE: A CONVENIENT SELECTION MARKER FOR THERMUS THERMOPHILUS. JBIOCHEM(TOKYO) 126, 951-956.

HUNGATE, R. E. (1969) A ROLL TUBE METHOS FOR CULTIVATION OF STRICT ANAEROBES. IN METHODS IN MICROBIOLOGY EDS. NORRIS, J. R. AND RIBBONS, D. W. PP.118-132. NEW YORK: ACADEMIC PRESS.

KOZIANOWSKI, G., CANGANELLA, F., RAINEY, F. A., HIPPE, H. AND ANTRANIKIAN, G. (1997) PURIFICATION AND CHARACTERIZATION OF THERMOSTABLE PECTATE-LYASES FROM A NEWLY ISOLATED THERMOPHILIC BACTERIUM, THERMOANAEROBACTER ITALICUS SP. NOV. EXTREMOPHILES 1, 171-182.

KUMAR, S., TAMURA, K., JAKOBSEN, I. B. AND NEI, M. (2001) MEGA2: MOLECULAR EVOLUTIONARY GENETICS ANALYSIS SOFTWARE. BIOINFORMATICS 17, 1244-1245.

LARSEN, L., NIELSEN, P. AND AHRING, B. K. (1997) THERMOANAEROBACTER MATHRANII SP. NOV., AN ETHANOL-PRODUCING, EXTREMELY THERMOPHILIC ANAEROBIC BACTERIUM FROM A HOT SPRING IN ICELAND. ARCHMICROBIOL 168, 114-119.

1. (canceled)

2. (canceled)

3. (canceled)

4. (canceled)

5. (canceled)

6. An isolated cell of strain BG10 (DSMZ Accession number 23015) or strain BG4 (DSMZ Accession Number 23012) or a mutant of any thereof, which cell or mutant has a decreased production of acetic acid after deletion of the genes encoding phosphotransacetylase and acetate kinase.

7. (canceled)

8. The isolated cell of 6, wherein the cell is capable of growing and producing fermentation products in a medium comprising a hydrolysed lignocellulosic biomass material having a dry-matter content of at least 10% wt/wt.

9. (canceled)

10. (canceled)

11. (canceled)

12. (canceled)

13. (canceled)

14. (canceled)

15. (canceled)

16. (canceled)

17. (canceled)

18. The isolated cell according to claim 6, wherein one or more genes have been inserted, deleted or substantially inactivated.

19. The isolated cell according to claim 18, wherein a gene encoding lactate dehydrogenase (LDH) (EC 1.1.1.27) has been down-regulated, deleted or substantially inactivated.

20. (canceled)

21. (canceled)

22. The isolated cell according to claim 19, which is BG10XL (DSMZ Accession number 23017) or BG4XL (DSMZ Accession number 23014).

23. The isolated cell according to claim 18, wherein a gene encoding an acetate kinase (EC 2.7.2.1) has been down-regulated, deleted or substantially inactivated.

24. (canceled)

25. (canceled)

26. The isolated cell according to claim 6, which is BG10pka (DSMZ Accession number 23216) or BG4pka (DSMZ Accession number 23013).

27. The isolated cell according to claim 18, wherein a gene encoding a phosphate acetyltransferase (EC 2.3.1.8) has been down-regulated, deleted or substantially inactivated.

28. (canceled)

29. (canceled)

30. The isolated cell according to claim 27, which is BG10pka (DSMZ Accession number 23216) or BG4pka (DSMZ Accession number 23013).

31. The isolated cell according to claim 18, wherein one or more genes have been inserted.

32. The isolated cell according to claim 31, wherein one or more genes encoding a polysaccharase has been inserted.

33. (canceled)

34. The isolated cell according to claim 31, wherein one or more genes encoding a pyruvate decarboxylase (EC 4.1.1.1) has been inserted.

35. The isolated cell according to claim 31, wherein one or more genes encoding an alcohol dehydrogenase (EC 1.1.1.1, EC 1.1.1.2, EC 1.1.1.71, EC 1.1.99.8) has been inserted.

36. The isolated cell according to claim 6, wherein the expression of one or more genes encoding an alcohol dehydrogenase (EC 1.1.1.1, EC 1.1.1.2, EC 1.1.1.71, EC 1.1.99.8) has been increased.

37. An isolated cell of strain BG10 (DSMZ Accession number 23015).

38. An isolated cell of strain BG4 (DSMZ Accession number 23012).

39. An isolated strain comprising a Thermoanaerobacter italicus cell according to claim 6.

40. A method of producing a fermentation product comprising culturing a cell according to claim 6 under suitable conditions.

41. (canceled)

42. (canceled)

43. (canceled)

44. (canceled)

45. A method according to claim 40, wherein the fermentation product is selected from the group consisting of an acid, an alcohol, a ketone and hydrogen.

46. (canceled)

47. (canceled)

48. (canceled)

49. (canceled)

50. (canceled)

51. A process for preparing a mutant Thermoanaerobacter italicus cell, comprising inserting, upregulating, deleting or substantially inactivating one or more genes in the isolated cell of any one of claim 6.
